(19) United States
(12) Patent Application Publication
(10) Pub. No.: US 2005/0131512 A1
(43) Pub. Date: Jun. 16, 2005

(54) Stent delivery catheter

(75) Inventor: Carlos Vonderwalde, Richmond (CA)

Correspondence Address: EDWARD J. LYNCH;JONES, TULLAR & COOPER, P.C., 2001 JEFFERSON DAVIS HIGHWAY, BOX 2266 EADS STATION, ARLINGTON, VA, 22202, US

(73) Assignee: Design & Performance-Cyprus Ltd

(21) Appl. No.: 10/735,548

(22) Filed: Dec. 12, 2003

(51) Int. Cl. A61F 2/06

(52) U.S. Cl. 623/1.11; 606/194

(57) ABSTRACT

The present invention is directed to a stent delivery catheter having a distal shaft section with an inflatable balloon and a first tubular portion or member extending though the interior of the balloon. A length of the first tubular portion extending through the interior of the balloon has an region with a surface which extends outwardly further than an adjacent region of the first tubular portion. This stepped surface facilitates securing the end of the stent mounted on the balloon and minimizes stent movement during handling and intracorporeal deployment. Preferably, the first tubular member has a proximal step-up region and a distal step-down region over which the ends of the stent are crimpled.

### BACKGROUND OF THE INVENTION

The present invention relates to the delivery of expandable endoprosthesis devices, generally called stents, which are adapted to be implanted into a patient's body lumen, such as a blood vessel, to maintain the patency thereof. Stents are particularly useful in the treatment and repair of blood vessels after or during percutaneous transluminal coronary angioplasty (PTCA), percutaneous transluminal angioplasty (PTA), or atherectomy and reduce the possibility of restenosis. They may also be employed to treat vulnerable plaque.

Stents are cylindrically shaped, usually metallic tubular devices which function to hold open and sometimes expand a segment of a blood vessel or other body lumen, such as coronary artery. Stents are usually delivered in a compressed condition to the target site and then deployed at that location into an expanded condition to support the vessel and help maintain it in an open position. They are also suitable for use to support and hold back a dissected arterial lining after an angioplasty procedure to avoid occlusion of the arterial passageway.

A variety of stent designs have been used. One of the difficulties encountered in prior art stents involve maintaining the radial rigidity needed to hold open a body lumen, while at the same time maintaining the longitudinal flexibility of the stent to facilitate its delivery and accommodate the often tortuous path of the body lumen.

PTCA is a well established minimally invasive procedure for the treatment of heart disease, wherein a balloon catheter is advanced within the patient's vasculature until the balloon on the catheter is disposed within the arterial blockage and the balloon is inflated to expand the blockage to thereby increase the blood flow therethrough. In a typical PTCA procedure, a guiding catheter is first percutaneously inserted into the patient's cardiovascular system either through the brachial or the femoral arteries, and is advanced until the distal tip of the guiding catheter is seated within the ostium of the desired coronary artery. A balloon dilatation catheter is then advanced out of the guiding catheter into a patient's coronary artery through the inner lumen of the guiding catheter, until the balloon at the distal portion of the catheter is disposed within the desired region of the patient's artery. The balloon is inflated and deflated one or more times as required to re-open the arterial passageway and thereby permit blood flow volume to increase once the catheter is removed.

Most angioplasty procedures today involve placement of a stent at the site to minimize restenosis and generally support the arterial region. Typically, the stent is delivered with the balloon expansion to dilate the stenosis. However, the stent may be delivered after the angioplasty procedure is completed with another balloon catheter which is similar to the balloon catheter used for the angioplasty procedure.

There are generally two types of catheters used in PTCA/stent delivery procedures, namely the rapid exchange type balloon catheters and the over-the-wire type balloon catheters. However, by far, most stents are delivered with rapid exchange type delivery catheters.

A rapid exchange type balloon catheter has a relatively short guide wire receiving lumen extending through a distal portion of the catheter with one guide wire port at the distal end of the catheter and another guide wire port spaced about 5 to about 50, usually about 10 to about 40 cm from the distal end. These catheters allow for the rapid exchange of the catheter without the need for an exchange wire or adding a guide wire extension to an in-place guide wire. Over-the-wire balloon catheters have guide wire lumens which extend the entire length of the catheter and require guide wire extensions or exchange wires to exchange the catheter.

Key features for effective stent delivery include delivery of the stent through tortuous anatomy without damage to the stent or displacement of the stent from the balloon. This requires that the catheter be highly responsive to the controlled advancement of the balloon catheter with optimal pushability from the proximal shaft section, and at the same time, retaining overall optimal flexibility for advancement within the tortuous anatomy of the patient's vascular structures.

There is a need for stent delivery catheter systems which provide superior pushability and flexibility and, and at the same time, be capable of properly supporting the collapsed stent on the working length of the balloon. The present invention satisfies these and other needs as will be apparent from the following disclosure when taken in conjunction with the accompanying exemplary drawings.

### SUMMARY OF THE INVENTION

The present invention is directed to an intracorporeal catheter and particularly to a stent delivery catheter. The catheter embodying features of the present invention has an elongated shaft with an inflation lumen and a guide wire lumen extending therein and an inflatable member or balloon which is disposed about a distal shaft section[and which is configured to expand a stent mounted on the exterior of the inflation member].

The distal shaft section includes a first tubular portion or member which has a length disposed within the interior of the inflatable member that has at least one and preferably at least two stepped regions with changes in transverse or radial dimensions at the ends of the length. For example, the length of the first tubular portion may be diametrically larger than one or more regions adjacent to the first tubular portion. The length of the inner tubular member serves as a raised portion or bumper over which the balloon and a mounted stent are positioned when the stent is crimped onto the balloon. The length of the inner tubular member is configured to be shorter than the stent to be mounted thereon, so that when the stent is crimped onto the balloon, the ends of the stent are crimped on the balloon over at least one stepped regions at the ends of the length of the inner tubular member. With this structure the stent is mounted very securely and is not likely to move on the balloon or be damaged when the stent delivery system is handled or advanced within the patient's vasculature and positioned for deployment.

The elongated catheter shaft has a proximal shaft section preferably with a proximal end, an inflation port in the proximal end and part of the inflation lumen extending to and in fluid communication with the inflation port in the proximal end. An adapter is provide on the proximal end to direct inflation fluid through the inflation port to the inflation lumen of the proximal shaft section.

The elongated catheter shaft may also have an intermediate shaft section which has part of the inflation lumen in fluid communication with the portion of the inflation lumen in the proximal shaft section and the portion of the inflation lumen in the distal shaft section to deliver inflation fluid to the inflatable member or balloon on the distal shaft section. The intermediate shaft section may also have of the guide wire lumen that is in fluid communication with the portion of the guide wire lumen in the first tubular portion. The guide wire lumen is configured to slidably receive a guide wire over which the catheter is advanced within the patient. The guide wire lumen extends the entire length of the catheter for over-the-wire type catheters and is relatively short for rapid exchange type catheters.

The distal shaft section is preferably relatively flexible to facilitate advancement through tortuous anatomy, the proximal shaft section relatively stiff to provide push and the intermediate shaft section acts as a transition between the proximal and distal shaft sections and to minimize kinking.

The inflation and guide wire lumens of the intermediate section may be of a concentric design in which an inner tubular member defines the guide wire lumen and an outer tubular member, disposed about the inner tubular member, defines an annular inflation lumen between the inner and outer tubular members. Alternatively, the intermediate shaft section may have a dual lumen construction in which the inflation lumen and the guide wire lumen are independent lumens in a side-by side or over and under locations within the intermediate shaft section.

An inflatable member, such as a balloon, has an elongated working section, preferably cylindrically shaped, disposed about the first tubular portion of the distal shaft section and extends over the length and the adjacent stepped regions of the inner tubular member. The balloon is provided with tapered ends adjacent to the working section which lead to skirts. The distal skirt of the balloon is sealingly secured to the distal end of the first tubular portion of the distal shaft section and the proximal skirt of the balloon is sealingly secured to the exterior of the shaft proximal to the balloon.

The stent is mounted on the working section of the balloon for delivery, usually by crimping, and is generally the same length or a little shorter than the working length of the balloon. However, the stent is generally longer than the larger diameter length of the inner tubular portion so that one or both ends of the stent are crimped onto a smaller diameter section adjacent to the larger diameter length.

The stent delivery catheter may be provided in either a rapid exchange design which has a proximal guide wire port a short distance, e.g. 5 to about 50, preferably about 10 to about 40 cm, from the distal end of the catheter or an over-the-wire design as described above in which the guide wire lumen extends the length of the catheter and is in fluid communication with the port in the proximal end of the catheter.

In a rapid exchange configuration, the proximal shaft section has a relatively stiff tubular member, preferably formed of hypotubing, with a polymeric jacket which extends beyond the distal end of the relatively stiff tube and is sealed to the intermediate shaft section to form a fluid passageway from the inner lumen of the relatively stiff tube to an inflation lumen in the intermediate shaft section. The inflation lumen is in fluid communication with the adaptor on the proximal end having a luer connector which is configured to be connected to a source of inflation fluid for the inflatable balloon. The relatively stiff proximal shaft section of the catheter provides imporved pushability. A reinforcing strut or wire may be secured by its proximal end to the interior of the distal portion of the hypotube. The free end of the strut or wire extends to a location such as within the inflation lumen of the intermediate shaft section of the catheter to transition the stiffness across the proximal guide wire port. The guide wire lumen in the intermediate shaft section leads to the proximal guide wire port which is disposed a short distance from the distal end of the catheter and which is preferably proximal to the free end of the reinforcing strut or wire. In one embodiment, the hypotube is provided with a polymeric exterior jacket which extends beyond the distal end of the hypotube and is sealingly secured to the intermediate shaft section to provide a sealed passageway between the inflation lumen of the hypotube and the inflation lumen of the intermediate shaft section.

The invention provides a stent delivery design with improved profiles and ease of use, particularly with covered stents. These and other advantages of the invention will become more apparent from the following detailed description of the invention and the accompanying exemplary drawings.

### BRIEF DESCRIPTION OF THE DRAWINGS

FIG. 1 is an elevational view of a balloon catheter, partially in section, which embodies features of the invention.

FIG. 2 is a transverse cross sectional view of the catheter shown in FIG. 1, taken along lines 2-2.

FIG. 3 is a transverse cross sectional view of the catheter shown in FIG. 1, taken along lines 3-3.

FIG. 4 is a transverse cross sectional view of the catheter shown in FIG. 1, taken along lines 4-4.

FIG. 5 is a schematic partial elevational view of the distal portion of the balloon catheter shown in FIG. 1 with a stent surrounding but not crimped on the balloon.

FIG. 5A is a longitudinal cross section of the distal portion of the balloon catheter shown in FIG. 1 with the stent crimped onto the balloon.

FIG. 6 is an elevational view of a stent with a stent cover having the ends thereof under the undulations of the end cylindrical sections of the stent.

FIG. 7 is an elevational view of part of the distal shaft section and part of the intermediate shaft section of an alternative stent delivery catheter embodying features of the invention, wherein the catheter shaft proximal to the balloon has two parallel lumens.

FIG. 8 is a transverse cross-sectional view of the catheter shown in FIG. 7 taken along the lines 8-8.

FIG. 9 is an elevational view, partially in section, of an over-the-wire balloon catheter embodying features of the invention.

FIG. 10 is a transverse cross sectional view of the catheter shown in FIG. 8, taken along lines 10-10.

FIGS. 11, 12 and 13 are elevational views of alternative tubular constructions for the distal shaft section within the interior of the inflatable member.

### DETAILED DESCRIPTION OF EMBODIMENTS OF THE INVENTION

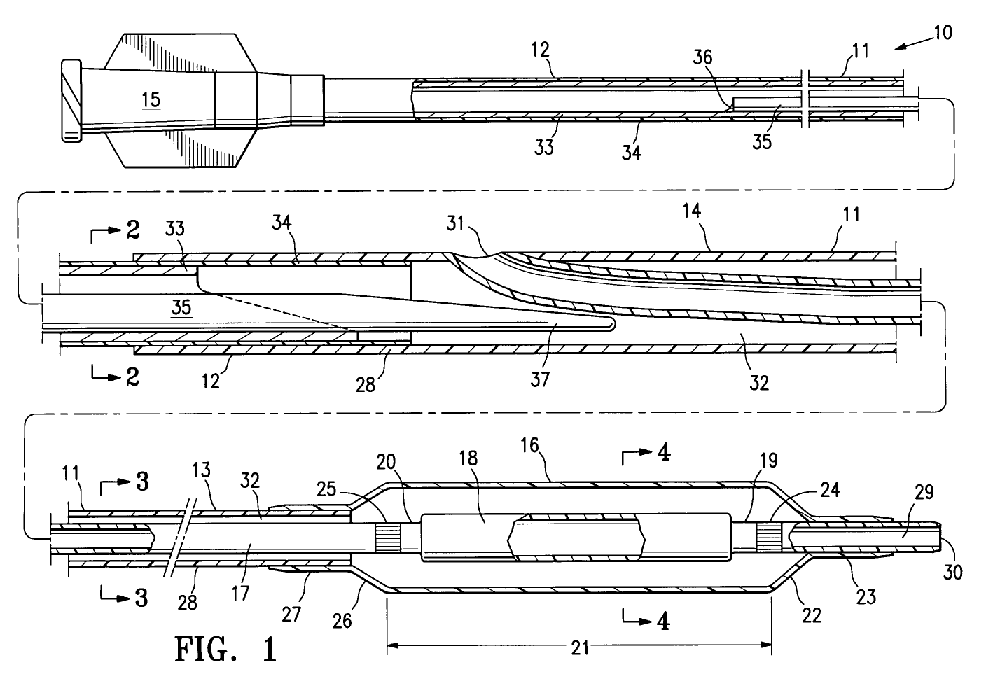

FIGS. 1-4 illustrates a stent delivery catheter 10 embodying features of the invention which includes an elongated shaft 11 having a relatively stiff proximal shaft portion 12, a flexible distal shaft section 13 and an intermediate shaft section 14, an adapter 15 mounted on the proximal end of the elongated shaft, and an inflatable balloon 16 on the distal shaft section.

The distal shaft section 13 has a first tubular portion or member 17 with a length 18 that is diametrically larger than diametrically smaller regions 19 and 20 which are respectively distally and proximally adjacent to length 18 to provide a step-up at the proximal end of the length and a step-down at the distal end of the length. Diametrically larger length 18 is positioned within the interior of the balloon 16 and is generally centered with respect to the working length 21 of balloon 16. The balloon 16 has a distal taper 22 leading from the distal end of working section 21 to distal skirt 23 which is sealingly secured to the exterior of the distal end of first tubular member 17. Radiopaque markers 24 and 25 are provided on the exterior of inner tubular portion 17 to mark the ends of the working length 21 of balloon 16. The balloon 16 also has a proximal taper 26 leading from the proximal end of working length 21 to proximal skirt 27 which is sealingly secured to outer tubular member 28. The inner tubular portion 17 has an inner lumen 29 which extends from distal guide wire port 30 in the distal end of first tubular portion to proximal guide wire port 31.

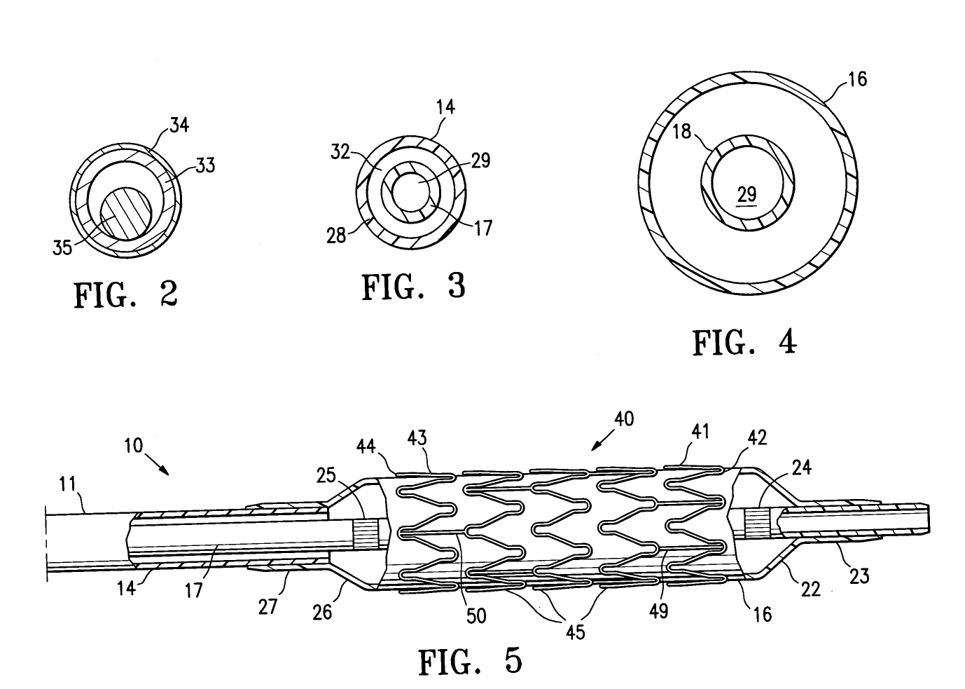

As shown in FIG. 2, the intermediate shaft section 14 has outer tubular member 28 concentrically disposed about first tubular portion 17 to form the annular inflation lumen 32 which is in fluid communication with the interior of balloon 16. The proximal end of inner tubular member 17 is curved to the exterior of outer tubular member 28 to form the proximal guide wire port 31.

The proximal shaft section 12 includes a high strength tubular member 33, such as a hypotube, with a polymeric outer jacket 34 which extends beyond the distal end of the tubular member 33 and is sealingly secured to the outer tubular member 28. A transitioning strut or support wire 35 is secured by its proximal end 36 to the interior of the tubular member 33 and the free distal end 37 thereof extends into the annular inflation lumen 32 defined between the first tubular member 17 and outer tubular member 28.

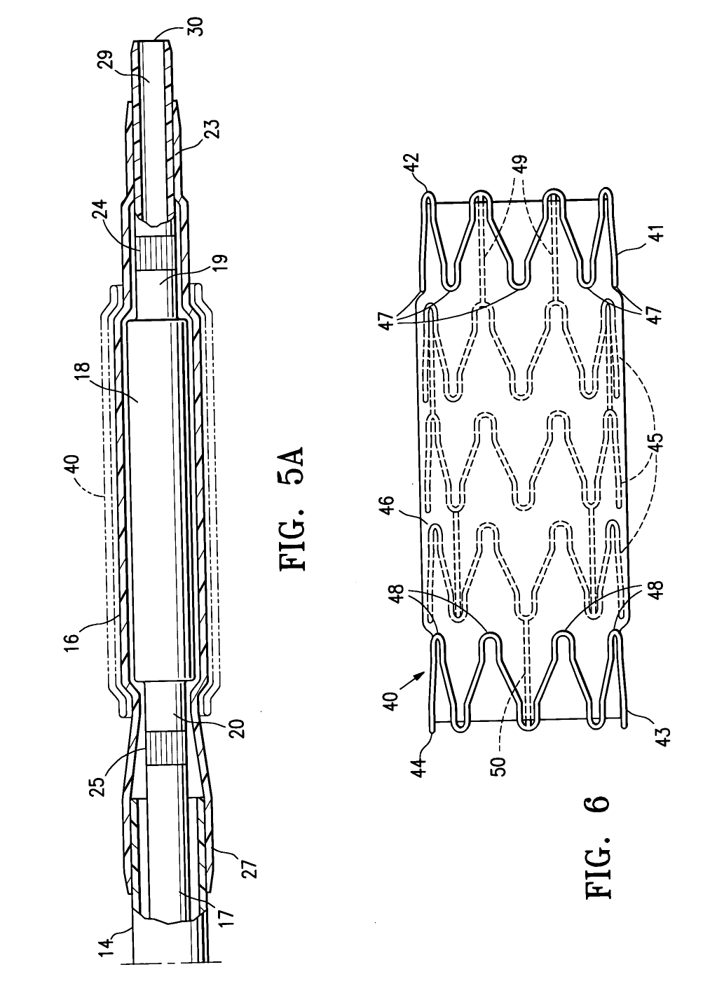

A suitable balloon expandable stent 40 is shown in FIG. 5 which is formed of a plurality of interconnected cylindrical wall sections including a first cylindrical wall section 41 at a first end 42 of the stent, a second cylindrical wall section 43 at a second end 44 of the stent and a plurality of intermediate cylindrical wall sections 45 between the first and second cylindrical walls 41 and 43. FIG. 5A illustrates the stent 40 (in phantom) crimped onto the balloon 16. As shown in FIG. 6 the stent 40 may be provided with a stent cover 46 which extends over the intermediate cylindrical wall sections 45 and which fits in part under the undulations 47 and 48 of the end cylindrical wall sections 41 and 43 and over the connecting members 49 and 50 as shown. Details of the stent 40 and stent cover 46 are described in co-pending application Ser. No. 09/664,999, filed Sep. 18, 2000, which is incorporated herein by reference in its entirety.

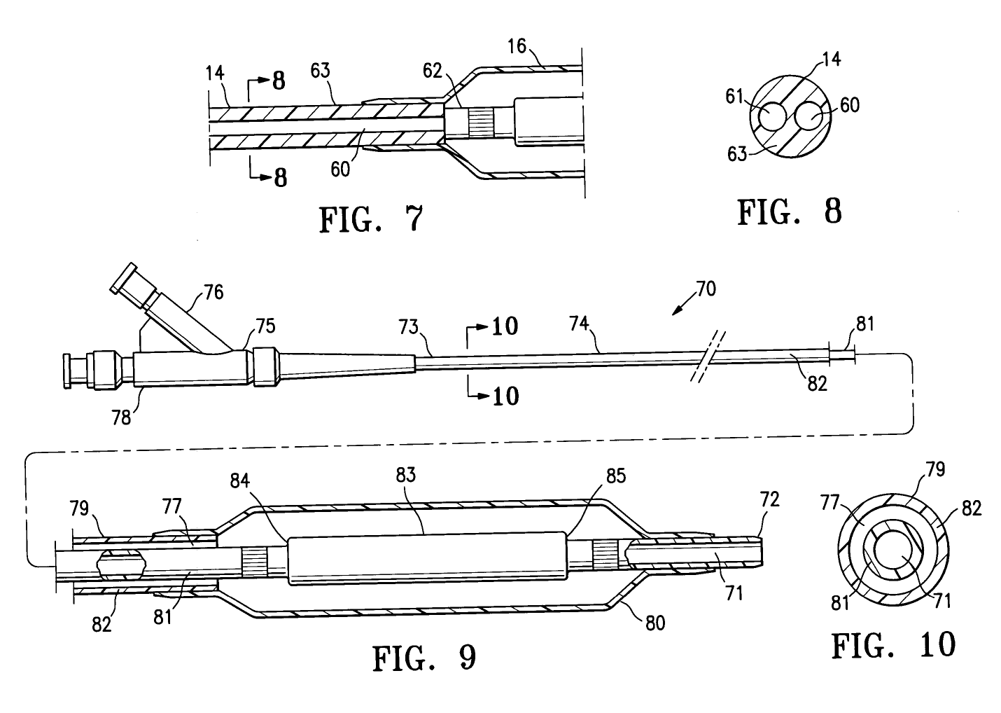

An alternate construction of the intermediate shaft section 14 is illustrated in FIGS. 7 and 8. In this alternate embodiment the intermediate shaft 14 is formed of a dual lumen construction with an inflation lumen 60 in fluid communication with the interior of balloon 16 and a guide wire lumen 61 which extends into the inner tubular member 62. The lumens 60 and 61 are off-set from one another. The intermediate shaft section 14 is shown in FIGS. 7 and 8 as a monolithic member 63. However, this shaft section may be formed of a first tubular member defining the inflation lumen, a second tubular member defining the guide wire lumen and an outer sheath. These members may be secured together by adhesive or fused together in a suitable manner.

An alternative stent delivery catheter 70 embodying features of the invention may also be provided in an over-the-wire configuration, as shown in FIGS. 9 and 10, in which the guide wire lumen 71 extends the length of the catheter 70 from the distal tip 72 to the proximal end 73 of the catheter shaft 74. A two-arm adapter 75 is shown mounted on the proximal end 73 of the catheter shaft 74 with one arm 76 for the introduction of inflation fluid to the inflation lumen 77 and one arm 78 for the introduction of a guide wire (not shown) into the guide wire lumen 71. The catheter shaft section 79 proximal to the inflatable balloon 80 is shown with inner tubular member 81 and outer tubular member 82 in a concentric construction. The length 83 of the inner tubular member 81 with proximal step 84 and distal step 85 is configured to receive the balloon 80 and a stent (not shown) with the ends of the stent crimped over the steps 84 and 85 as shown in FIG. 5A. An alternative dual lumen construction shown in FIGS. 7 and 9 may also be employed.

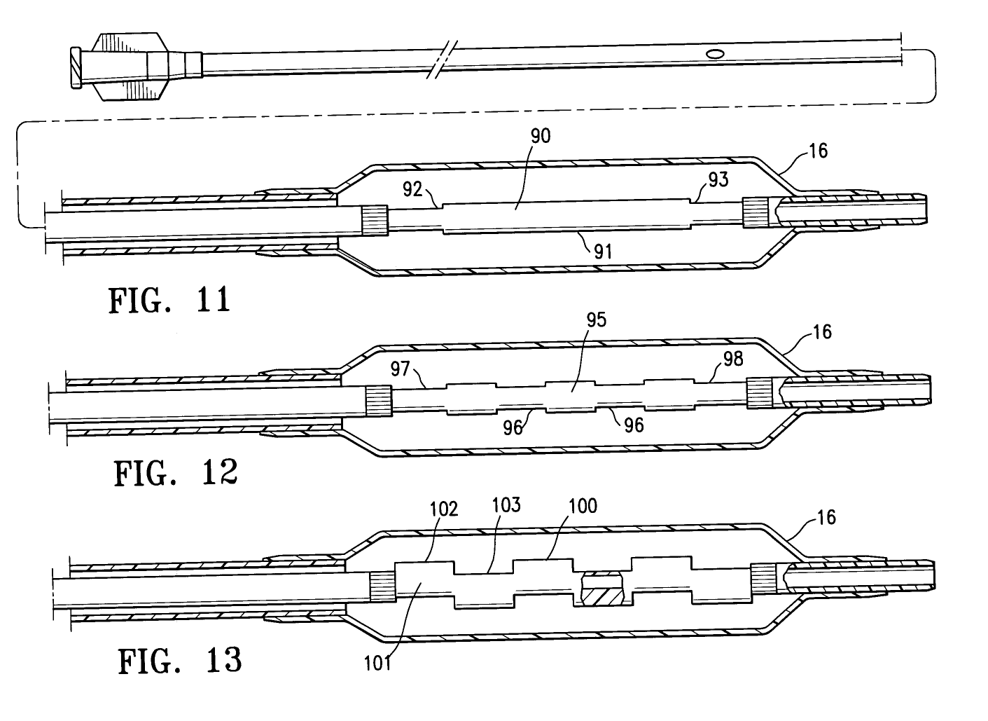

FIGS. 11, 12 and 13 illustrate alternative structures for the portion of the inner tubular member 90 which extends through the interior of a balloon 16. In FIG. 11 the outer diameter of the diametrically larger length 91 generally the outer diameter of the inner tubular member and the reduced diameters of the stepped regions 92 and 93 are recesses in the exterior of the inner tubular member. The diametrically larger length 95 of the alternative structure shown in FIG. 12 itself has diametrically smaller regions 96 in addition to the stepped regions 97 and 98 at the ends of the length 95. In the embodiment shown in FIG. 13 the length 100 of the first tubular member 101 over which the stent is to be crimped has alternating stepped regions, one stepped region 102 of the surface extends out radially more than an adjacent region 103 of the inner tubular member. In each of these alternative embodiments, the ends of the stent are crimped onto a balloon surrounding and preferably folded over the stepped ends of the first tubular portion when the stent is crimped onto the balloon.

The catheter components may be formed of conventional materials used in angioplasty and stent delivery catheters and may be formed in a conventional manner. Multilayered components are anticipated. The elongated catheter shaft will generally have the dimensions of conventional dilatation or stent delivery catheters. The length of the catheter, measured from the distal end of the adapter 14 to the distal end of inner tubular member 17 of the elongated catheter shaft 11 may be about 90 cm to about 150 cm, and typically, it is about 137 cm.

The material composition for the outer tubular member may be formed from nylon polymeric material such as PEBAX® polymers (Elf Atochem North America Inc., Philadelphia, Pa.). Alternatively, the member may be made from families of polymers such as polyamides, polyurethanes and polyesters. The polymer composite selected will be compatible with the polymeric material of the catheter component to which it is to be secured, preferably the composite are formed of the same material to facilitate adhesion to one another. Other suitable polymer materials include polyolefin based copolymer with reactive monomer forming the copolymer. A presently preferred polyolefinic material is a polyethylene based adhesive polymer such as ethylene-acrylic acid copolymer sold commercially as PRIMACOR (Dow Chemical Co.), ESCOR (Exxon), or PLEXAR (Quantum Chemical Corp.).

The balloon may be made from Nylon, polyethylene, polyethylene terepthalate, polyurethanes, polyamide/polyether block copolymers (optionally linked with amide or ester linkages), polyether block amide such as PEBAX 70 (Elf Atochem), and other relatively inelastic polymers or other materials known in the art. The working length of the balloon for coronary artery use may be about 10 mm to about 50 mm, preferably about 10 mm to about 40 mm. The balloon diameter for coronary catheters is generally about 0.5 mm to about 5 mm, typically about 1.5 mm to about 4.5 mm in the inflated condition. The dimensions for balloons for peripheral artery use generally may be larger. The wall thickness of the balloon will depend on the selected burst pressure requirements and the hoop strength of the balloon material selected, but generally ranges from about 0.00025 to about 0.00045 inch (0.006-0.011 mm).and typically about 0.00035 inch (0.009 mm). The balloon tapers 22 and 26 are about 4 to about 6 mm and typically about 5 mm, as measured along the length of the inner tubular member 17. The cone angle for the balloon tapers are about 15° to about 50°, preferably about 20° to about 45°. The balloon skirts 23 and 27 are about 1 to about 5 mm in length, preferably about 1.5 to about 3 mm, with diameters of about 0.025 to about 0.045 inch (0.64 mm-1.1 mm), typically about 0.033 to about 0.37 inch (0.8 mm-0.9 mm). The distal skirt diameter is slightly smaller than the proximal skirt diameter.

The diametrically enlarged portion of the first tubular portion has a length of about 5 to about 15 mm less than the working length of the balloon and a diameter of about 0.7 to about 1.4 mm, preferably about 0.9 to about 1.2 mm and typically about 1.1 mm. The reduced diameter regions adjacent to the diametrically larger length 18 are about 1 to about 4 mm, preferably about 1.5 to about 3.5 mm in length and are about 0.05 to about 0.4 mm preferably about 0.1 to about 0.3 mm less than the diameter of the diametrically larger length 18.

The first tubular portion may be formed of suitable polymeric materials such as polyamides, polyurethanes, polyesters and polyethylene based polymers. The exterior of the tubular member should be compatible with the balloon material to facilitate thee bonding thereof. The interior is preferably formed of a lubricious material such as polyethylene based polymers to facilitate guide wire movement within the inner lumen of the inner tubular member. The guide wire lumen of the first tubular portion has a diameter which should be configured to allow for the passage of conventional guide wires to be used in these types of procedures. Preferably the diameter of the inner lumen should be about 0.001 to about 0.005 inch (0.03 mm-0.13 mm) larger than the guide wire diameter.

The outer tubular member is formed of a polymeric material which is compatible with the balloon material. For example, for a Nylon balloon, PEBAX polymer (Elf Atochem North America, Inc.) is a suitable material for the outer tubular member. The outer diameter of the outer tubular member is configured to fit in the proximal skirt The wall thickness should be adequate to provide suitable wall strength and provide an adequately sized annular inflation lumen between the inner and outer tubular members.

The hypotube of the proximal shaft section may be formed of a metallic material, and is preferably formed of a metallic material selected from the group consisting of 304 v stainless steel, superelastic NiTi alloy, MP35N, Elgiloy, and related alloy materials. Non-metallic materials may also be used to form the hypotube, including braided polyimide, high strength polymers such as polyetheretherketone (PEEK), polyetherketone, and polyketone.

The length of the hypotube is generally about 80 cm to about 120 cm, preferably about 90 to about 110 cm, for coronary applications with a wall thickness of about 0.003 to about 0.01 inch (0.08-0.25 mm), preferably about 0.004 to about 0.006 inch (0.1-0.15 mm).

Generally, the use of the stent delivery catheters described herein would typically be as follows. A guiding catheter (not shown) is advanced through the patient's vasculature until the distal end thereof is located adjacent to the ostium of the coronary artery. The proximal end of the guiding catheter is torqued to seat the distal end of the guiding catheter into ostium of the coronary artery. The stent delivery balloon catheter is prepared for insertion by advancing the stent over the catheter until the stent is positioned over the working length of the balloon and then crimped thereon. The ends of the stent are crimped onto the reduced diameter portion adjacent to the diametrically enlarged portion of the inner tubular portion. A guide wire is inserted into the guiding catheter and advanced therein until the distal tip of the guide wire is distal to the stent deployment site. The balloon catheter with mounted stent is advanced over the guide wire in the patient's vascular system, until the balloon and the stent mounted thereon are located across the desired region of the body lumen. The balloon may then be inflated in a conventional manner known in the art by introducing and pressurizing radiopaque liquid or other inflation fluid. After the balloon has been inflated and the stent deployed, the balloon can be deflated and the catheter removed. Inflation-deflation may be repeated multiple times as necessary to effect the desired expansion and placement of the stent. Once the stent is properly placed, the balloon may be deflated and the catheter removed from the patient.

While particular forms of the invention have been illustrated and described, it will be apparent that various modifications and improvements can be made to the invention. For example, the stent may be coated or provided with a jacket having one or more therapeutic or diagnostic agent incorporated therein Unless described otherwise, conventional materials and methods of construction may be used to make the catheters and stents. Although individual features of embodiments of the invention may be shown in some drawings and not in others, those skilled in the art will recognize that individual features of one embodiment of the invention can be combined with any or all the features of another embodiment. Accordingly, it is not intended that the invention be limited to the specific embodiments illustrated. It is therefore intended that this invention to be defined by the scope of the appended claims as broadly as the prior art will permit.

Terms such a “element”, “member”, “device”, “sections”, “portion”, “section”, “steps”, “means” and words of similar import when used herein shall not be construed as invoking the provisions of 35 U.S.C. §112(6) unless the following claims expressly use the terms “means” followed by a particular function without specific structure or “step” followed by a particular function without specific action. All patents and patent applications referred to above are hereby incorporated by reference in their entirety.

1. A stent delivery catheter having an elongated catheter shaft with a guide wire lumen and an inflation lumen, comprising:
a. a distal shaft section which has a first tubular portion defining in part the guide wire lumen, having a distal end and a guide wire port in the distal end in fluid communication with the guide wire lumen, having at least one stepped exterior region over which an end of a stent may be crimped; and
b. an inflatable member mounted on the distal shaft section which has an interior in fluid communication with the inflation lumen, which extends over the first tubular portion with the stepped exterior region, which has a distal portion sealingly secured to the distal end of the first tubular portion and which has a working length configured to receive a stent thereon.

2. The stent delivery catheter of claim 1 wherein the portion of the first tubular portion over which the inflatable member extends has two stepped regions.

3. The stent delivery catheter of claim 2 wherein one of the stepped regions is a step-up region and one is a step-down region.

4. The stent delivery catheter of claim 3 wherein the step-down region is distal to the step-up region.

5. The stent delivery catheter of claim 4 wherein the step-down region is aligned with a distal end of the working length of the inflatable member and the step-up region is aligned with the proximal end of the working length of the inflatable member.

6. The stent delivery catheter of claim 1, wherein a length of the first tubular portion over which the inflatable member extends has transverse dimensions greater than regions of the first tubular portion proximal and distal to the length.

7. The stent delivery catheter of claim 6 wherein the length of the first tubular portion having greater transverse dimensions is aligned with the working length of the inflatable member.

8. The stent delivery catheter of claim 1 including a proximal shaft section formed in part of a second tubular portion having a proximal end with an inflation port therein and a portion of the inflation lumen extending therein to and in fluid communication with the inflation port.

9. The stent delivery catheter of claim 8 including an intermediate shaft section between the proximal and distal shaft sections having part of the inflation lumen in fluid communication with the portion of the inflation lumen in the second tubular portion of the proximal shaft section and having part of the guide wire lumen in fluid communication with the portion of the guide wire lumen in the first tubular member of the distal shaft section.

10. The stent delivery catheter of claim 6 wherein the length of the first tubular portion has transverse dimensions of about 0.05 to about 0.5 mm greater than transverse dimensions of the adjacent regions thereof.

11. The stent delivery catheter of claim 6 wherein the length of the first tubular portion has transverse dimensions of about 0.1 to about 0.3 mm greater than the transverse dimensions of the adjacent regions thereof.

12. The stent delivery catheter of claim 1 wherein the length of the first tubular portion with greater transverse dimensions than adjacent regions thereof has a length ranging from about 5 to about 15 mm less than the working length of the inflatable member.

13. The stent delivery catheter of claim 1 wherein a proximal guide wire port is provided which is spaced a short distance proximally from the inflatable member and a substantial distance from the proximal end of the catheter and which is in fluid communication with the guide wire lumen.

14. The stent delivery catheter of claim 13 wherein the proximal guide wire port is spaced about 5 to about 50 cm from the distal end of the catheter.

15. The stent delivery catheter of claim 13 wherein the proximal guide wire port is spaced about 10 to about 40 cm from the distal end of the catheter.

16. The stent delivery catheter of claim 8 wherein the proximal shaft section is formed in part of hypotubing.

17. The stent delivery catheter of claim 8 wherein a transition member extends from a distal portion of the second tubular member of the proximal shaft section to a location in the inflation distal to the proximal guide wire port.

18. A rapid exchange type stent delivery catheter having an elongated catheter shaft with a guide wire lumen and an inflation lumen extending therein, comprising:
a. a distal shaft section which has a first tubular portion defining in part the guide wire lumen, having a distal end and a guide wire port in the distal end in fluid communication with the guide wire lumen, having at least one stepped exterior region over which an end of a stent may be crimped;
b. an inflatable member mounted on the distal shaft section which has an interior in fluid communication with the inflation lumen, which extends over the first tubular portion with the stepped exterior region; and
c. a proximal guide wire port in the elongated shaft which is disposed a short distance proximal to the inflatable member and a substantial distance from the proximal end of the catheter and which is in fluid communication with the guide wire lumen.

19. The stent delivery catheter of claim 18 including a proximal shaft section having a proximal end with a proximal inflation port therein and a portion of the inflation lumen extending therein to and in fluid communication with the inflation port.

20. The stent delivery catheter of claim 19 including an intermediate shaft section which is disposed between the proximal and distal shaft sections, which has part of the inflation lumen, which has part of the guide wire lumen and which has the proximal guide wire port.

21. The stent delivery catheter of claim 18 wherein the proximal guide wire port is spaced about 5 to about 50 cm from the distal guide wire port.

22. The stent delivery catheter of claim 18 wherein the proximal guide wire port is spaced about 10 to about 40 cm from the distal guide wire port.

23. A stent delivery system, comprising
a. a stent delivery catheter having an elongated catheter shaft with a guide wire lumen and an inflation lumen extending therein, comprising:
i. a distal shaft section which has a first tubular portion defining in part the guide wire lumen, having a distal end and a guide wire port in the distal end in fluid communication with the guide wire lumen, having at least one stepped exterior region over which an end of a stent may be crimped, and
ii. an inflatable member mounted on the distal shaft section which has an interior in fluid communication with the inflation lumen, which extends over the first tubular portion with the stepped exterior region, which has a distal portion sealingly secured to the distal end of the first tubular portion and which has a working length; and
b. a tubular stent crimped on the working length of the inflatable member with at least one end of the stent crimped over one of the stepped regions of the first tubular portion.

24. The stent delivery system of claim 23 wherein a second end of the stent is crimped over a second stepped region of the first tubular portion.

25. The stent delivery system of claim 23 wherein the catheter has a proximal shaft section, a proximal end with an inflation port therein and a portion of the inflation lumen extending therein to and in fluid communication with the inflation port.

26. The stent delivery system of claim 23 wherein the catheter has an intermediate shaft section between the proximal and distal shaft sections.

27. The stent delivery system of claim 26 wherein the intermediate shaft section has part of the inflation lumen in fluid communication with the portion of the inflation lumen in the proximal shaft section.

28. The stent delivery system of claim 26 wherein the intermediate shaft section has part of the guide wire lumen in fluid communication with the portion of the guide wire lumen in the first tubular portion.

29. The stent delivery system of claim 23 wherein the intermediate shaft section has a proximal guide wire port.

30. The stent delivery system of claim 29 wherein the proximal guide wire port is spaced about 5 to about 50 cm from the distal guide wire port.

31. The stent delivery system of claim 23 wherein the proximal guide wire port is spaced about 10 to about 40 cm from the distal guide wire port.

32. The stent delivery system of claim 23 wherein the length of the first tubular portion has a transverse dimension of about 0.05 to about 0.5 mm greater than transverse dimensions of adjacent regions thereof.

33. The stent delivery catheter of claim 23 wherein the length of the first tubular portion has a transverse dimension of about 0.1 to about 0.3 mm greater than the transverse dimension of the reduced diametrical regions adjacent to the length.

34. The stent delivery system of claim 23 wherein the length of the first tubular portion is about 5 to about 15 mm less than a working length of the balloon.

35. A stent delivery catheter having an elongated catheter shaft with a guide wire lumen and an inflation lumen, comprising:
a. a distal shaft section which has a first tubular member defining in part the guide wire lumen, a guide wire port in a distal end of the inner tubular member in fluid communication with the guide wire lumen and having a length that has at least one region along a length of the inner tubular member having a surface which extends radially further than a surface of an adjacent region of the inner tubular member to facilitate securely crimping a stent;
b. a proximal shaft section having a proximal end with an inflation port therein and a portion of the inflation lumen extending therein to and in fluid communication with the inflation port;
c. an intermediate shaft section between the proximal and distal shaft sections having part of the inflation lumen in fluid communication with the portion of the inflation lumen in the proximal shaft section and having part of the guide wire lumen in fluid communication with the portion of the guide wire lumen in the inner tubular member of the distal shaft section; and
d an inflatable member mounted on the distal shaft section which has an interior in fluid communication with the inflation lumen in the intermediate shaft section, which extends over the length of the first tubular member having a region with a surface extending radially further than the adjacent region thereof, which has a distal portion sealingly secured to the inner tubular member of the distal shaft section at a location distal to the length of the inner tubular member with the radially extending surface of the inner tubular member.

36. A stent delivery catheter having an elongated catheter shaft with a guide wire lumen and an inflation lumen, comprising:
a. a distal shaft section which has a first tubular member defining in part the guide wire lumen, a guide wire port in a distal end of the first tubular member in fluid communication with the portion of the guide wire lumen therein and having a length that is diametrically larger than regions of the first tubular member distally and proximally adjacent thereto;
b. a proximal shaft section having a proximal end with an inflation port therein and a portion of the inflation lumen extending therein to and in fluid communication with the inflation port; and
c. an inflatable member sealingly mounted on the distal shaft section which has an interior in fluid communication with the inflation lumen and which extends over the diametrically larger length of the first tubular member and the adjacent diametrically smaller regions thereof.

37. The stent delivery catheter of claim 36 wherein the diametrically larger length of the first tubular member has a transverse dimension of about 0.05 to about 0.5 mm greater than the transverse dimensions of the regions adjacent thereto.

38. The stent delivery catheter of claim 36 wherein the length of the first tubular member has a transverse dimension of about 0.1 to about 0.3 mm greater than the transverse dimension of the regions adjacent thereto.

39. An intracorporeal balloon catheter having an elongated catheter shaft with a guide wire lumen and an inflation lumen, comprising:
a. a distal shaft section which has a first tubular member defining in part the guide wire lumen, a guide wire port in a distal end in fluid communication with the guide wire lumen and having a length that is diametrically larger than regions of the inner tubular member distally and proximally adjacent thereto; and
b. an inflatable balloon member mounted on the distal shaft section which has an interior in fluid communication with the inflation lumen, which extends over the length of the first tubular member and the regions adjacent thereto, which has a distal portion sealingly secured to the distal end of the first tubular member and which has a working length extending over the length of the first tubular member and regions adjacent thereto.

40. A method of mounting a stent onto a stent delivery catheter comprising:
a. providing a stent delivery catheter having an elongated catheter shaft with a guide wire lumen and an inflation lumen extending therein, comprising:
i. a distal shaft section which has a first tubular portion defining in part the guide wire lumen, having a distal end and a guide wire port in the distal end in fluid communication with the guide wire lumen, having at least one stepped exterior region over which an end of a stent may be crimped, and
ii. an inflatable member mounted on the distal shaft section which has an interior in fluid communication with the inflation lumen, which extends over the first tubular portion with the stepped exterior region, which has a distal portion sealingly secured to the distal end of the first tubular portion and which has a working length;
b. sliding a tubular stent over the stent delivery catheter until the stent is disposed over the working length of the balloon; and
c. crimping the tubular stent onto the working length of the balloon with at least one end of the stent crimped over one of the stepped regions of the first tubular portion.
